(19) United States
(12) Patent Application Publication
(10) Pub. No.: US 2010/0233167 A1
(43) Pub. Date: Sep. 16, 2010

(54) CHAIN REACTION CREATING OLIGOMERS FROM REPEAT UNITS OF BINDING MOLECULES

(76) Inventors: Ramesh Bhatt, Belmont, CA (US); Lawrence Horowitz, Atherton, CA (US)

Correspondence Address: ARNOLD & PORTER LLP;ATTN: IP DOCKETING DEPT., 555 TWELFTH STREET, N.W., WASHINGTON, DC, 20004-1206, US

(21) Appl. No.: 12/597,948

(22) PCT Filed: May 9, 2008
(86) PCT No.: PCT/US08/63267

§ 371 Date: Mar. 25, 2010

Related U.S. Application Data
(60) Provisional application No. 60/917,294, filed on May 10, 2007.

(51) Int. Cl. A61K 39/395; C07K 16/46; A61P 35/00

(52) U.S. Cl. 424/134.1; 530/387.3

(57) ABSTRACT

The present invention concerns a chain reaction of cross-linking antibodies or other binding molecules prior or subsequent to binding to a target, such as a target antigen. The invention further concerns oligomers comprising repeat units of binding molecules, such as antibodies, optionally bound to a target, such as a target antigen. The invention also relates to antibodies and other binding molecules with multiple specificities useful in the methods of the invention, as well as various uses of the oligomers and individual binding molecules present in the oligomers.

### FIELD OF THE INVENTION

The present invention concerns a chain reaction of cross-linking antibodies or other binding molecules prior or subsequent to binding to a target, such as a target antigen. The invention further concerns oligomers comprising repeat units of binding molecules, such as antibodies, optionally bound to a target, such as a target antigen. The invention also relates to antibodies and other binding molecules with multiple specificities useful in the methods of the invention, as well as various uses of the oligomers and individual binding molecules present in the oligomers.

### BACKGROUND OF THE INVENTION

Antibodies have been used in a wide range of clinical applications, including in vitro and in vivo immunodiagnosis and therapy of a variety of diseases, including cancer. Thus, monoclonal antibodies have been approved and are in clinical use for the treatment of breast cancer (e.g., HERCEPTIN®, trastuzumab), non-Hodgkin lymphoma (e.g., RITUXAN®, rituximab), colorectal cancer (e.g., AVASTIN®, bevacizumab; ERBITUX®, cetuximab; VECTIBIX®, panitumumab), etc. Similarly, immunoadhesins, such as TNFR-Ig and CD4-Ig fusion proteins have shown efficacy in the treatment of various diseases, such as rheumatoid arthritis and HIV infection.

Monoclonal antibodies may achieve their therapeutic effect through various mechanisms, including, for example, cytotoxicity, apoptosis or programmed cell death and growth inhibition, e.g. by blocking growth factor receptors resulting in the arrest of proliferation of tumor cells. In cells that express monoclonal antibodies, monoclonal antibody therapy can rely on the formation of anti-idiotype antibodies. In addition, the efficacy of antibody therapy is often enhanced by other indirect mechanisms, such as recruiting cells with cytotoxic properties, such as monocytes and macrophages. Thus, antibodies can interact, through their Fc region, with Fc receptors present on monocytes, macrophages and/or natural killer cells (NK cells). This type of antibody-mediated cell kill is called antibody-dependent cell mediated cytotoxicity (ADCC). Monoclonal antibodies also bind complement, leading to direct cell toxicity, known as complement dependent cytotoxicity (CDC).

ADCC and/or CDC have been shown to play a major role in the mechanism of action of several therapeutic antibodies, such as HERCEPTIN® (trastuzumab, Genentech, Inc.) and RITUXAN® (rituximab, Biogen Idec, Inc.) (see, e.g., Eccles, S. A., Breast Cancer Res. 3:86-90 (2001)). Thus, enhancement of antibody effector functions, such as ADCC and/or CDC, is expected to result in improved efficacy in the treatment of viral infections, cancer therapy, and other indication areas.

In view of the emergence and promise of therapies based on antibodies and antibody-like molecules, there is a great need for approaches improving the efficacy and safety of such treatments. Similarly, there is a great need for new approaches to treat malignancies and other diseases or conditions benefiting from immunotherapy or the enhancement of immune clearance.

### SUMMARY OF THE INVENTION

In one aspect, the invention concerns a method for preparing an oligomer comprising repeats of a first and a second binding polypeptide bound to a target, comprising contacting molecules of

(a) the first binding polypeptide having binding specificity for the target and the second binding polypeptide having binding specificity for the first binding polypeptide, or

(b) the first binding polypeptide having binding specificity for a target and the second binding polypeptide having binding specificity for a complex formed between the first binding polypeptide and the target,

under conditions that a chain reaction between molecules of the first and the second binding polypeptides occurs,

wherein at least one molecule of the first binding polypeptide binds to the target before or after said chain reaction,

whereby an oligomer comprising repeats of the first and second binding polypeptides attached to the target at least one point is formed.

Conditions that facilitate chain reaction are those restricting intramolecular or bimolecular binding and allowing intermolecular or greater than bimolecular binding, such that a processive intermolecular chain reaction between molecules of the first and second binding polypeptides occurs.

In various embodiments, the first and second binding polypeptides are selected from the group consisting of antibodies, antibody fragments, surrogate light chain constructs, immunoadhesins, receptors, ligands, and enzymes.

In a preferred embodiment, the first and said second binding polypeptides are antibodies or antibody fragments and the target is an antigen. The antibody fragment may, for example, be selected from the group consisting of Fab, Fab′, F(ab′)₂, scFv, (scFv)₂, dAb, and complementarity determining region (CDR) fragments, linear antibodies, single-chain antibody molecules, minibodies, diabodies, and multispecific antibodies formed from antibody fragments.

In another embodiment, the second antibody or antibody fragment binds to the framework region of the first antibody or antibody fragment. In this embodiment, the oligomer formed is attached to the antigen at more than one point.

In yet another embodiment, the second antibody or antibody fragment binds to the antigen-binding region of the first antibody or antibody fragment. In this embodiment, the oligomer formed is attached to the antigen at one point (or just a few points).

In a further embodiment, the second antibody or antibody fragment binds to the complex formed between the first antibody or antibody fragment and the antigen. In this embodiment, the oligomer formed is attached to the antigen at more than one point.

In another aspect, the invention concerns a method for preparing an oligomer comprising repeats of a binding polypeptide having at least a first and second binding specificity, bound to a target, comprising contacting molecules of the binding polypeptide under conditions that restrict intramolecular or bimolecular binding and allow intermolecular, or greater than bimolecular binding such that a processive intermolecular chain reaction between molecules of the polypeptide occurs, wherein

(a) the first binding specificity is for a target and the second binding specificity is for another molecule of said binding polypeptide, or

(b) the first binding specificity is for a target and the second binding specificity is for a complex formed between said binding polypeptide and said target,

wherein at least one molecule of the binding polypeptide binds to the target before or after the chain reaction, and

whereby an oligomer comprising repeats of the binding polypeptide attached to the target is formed.

Just as before, the binding polypeptide may be selected from the group consisting of antibodies, antibody fragments, surrogate light chain constructs, immunoadhesins, receptors, ligands, and enzymes, for example. In a preferred embodiment, the binding polypeptide is a bi- or multi-specific antibody or an antibody fragment, and the target is an antigen, where the antibody fragment may, for example, be selected from the group consisting of Fab, Fab′, F(ab′)₂, scFv, (scFv)₂, dAb, and complementarity determining region (CDR) fragments, linear antibodies, single-chain antibody molecules, minibodies, diabodies, and multispecific antibodies formed from antibody fragments.

In one embodiment, the second binding specificity of the antibody or antibody fragment is for the framework region of another molecule of the antibody or antibody fragment. In this embodiment, the oligomer formed is attached to the antigen at more than one point.

In another embodiment, the second binding specificity of the antibody or antibody fragment is for the antigen-binding region of another molecule of the antibody or antibody fragment. In this embodiment, the oligomer formed is attached to the antigen at one point.

In yet another embodiment, the second binding specificity of the antibody or antibody fragment is for the complex formed between another molecule of the antibody or antibody fragment and the antigen. In this embodiment, the oligomer formed is attached to the antigen at more than one point.

In a further aspect, the invention concerns a bispecific polypeptide comprising a first binding region binding to a target and a second binding region recognizing and binding to a sequence within another molecule of the same bispecific polypeptide. In a particular embodiment, the bispecific polypeptide is a bispecific antibody or a bispecific antibody fragment.

In a still further aspect, the invention concerns an oligomer comprising repeats of a first and a second binding polypeptide bound to a target at least one site.

In a different aspect, the invention concerns a composition comprising a bispecific polypeptide or an oligomer, as described above, in admixture with a carrier. The composition may be a pharmaceutical composition.

In another aspect, the invention concerns a method for the prevention or treatment of a disease or condition benefiting from the enhancement of immune response comprising administering to a mammalian patient in danger of developing or having such disease or condition an effective amount of an oligomer described above.

The disease or condition may, for example, be a B cell neoplasm, such as a B cell lymphoma, including, for example, non-Hodgkin's lymphoma (NHL); follicular center cell (FCC) lymphoma, acute lymphocytic leukemia (ALL), chronic lymphocytic leukemia (CLL), and Hairy cell leukemia.

The non-Hodgkins lymphoma can, for example, be selected from the group consisting of low grade/follicular non-Hodgkin's lymphoma (NHL), small lymphocytic (SL) NHL, intermediate grade/follicular NHL, intermediate grade diffuse NHL, high grade immunoblastic NHL, high grade lymphoblastic NHL, high grade small non-cleaved cell NHL, bulky disease NHL, plasmacytoid lymphocytic lymphoma, mantle cell lymphoma, AIDS-related lymphoma and Waldenstrom's macroglobulinemia.

In another embodiment, the disease or condition benefiting from the enhancement of immune response is any type of malignancy, including solid tumors, such as breast cancer, colon cancer, pancreatic cancer, colon cancer, head and neck cancer, lung cancer, renal cancer, and the like, including metastatic cancers and cancers not responding or not responding well to existing therapies.

### BRIEF DESCRIPTION OF THE DRAWINGS

FIG. 1 illustrates an embodiment of the antibody chain reaction by showing the sequential binding of bispecific antibodies with binding specificity for a target antigen and the framework of another molecule of the antigen-bound antibody.

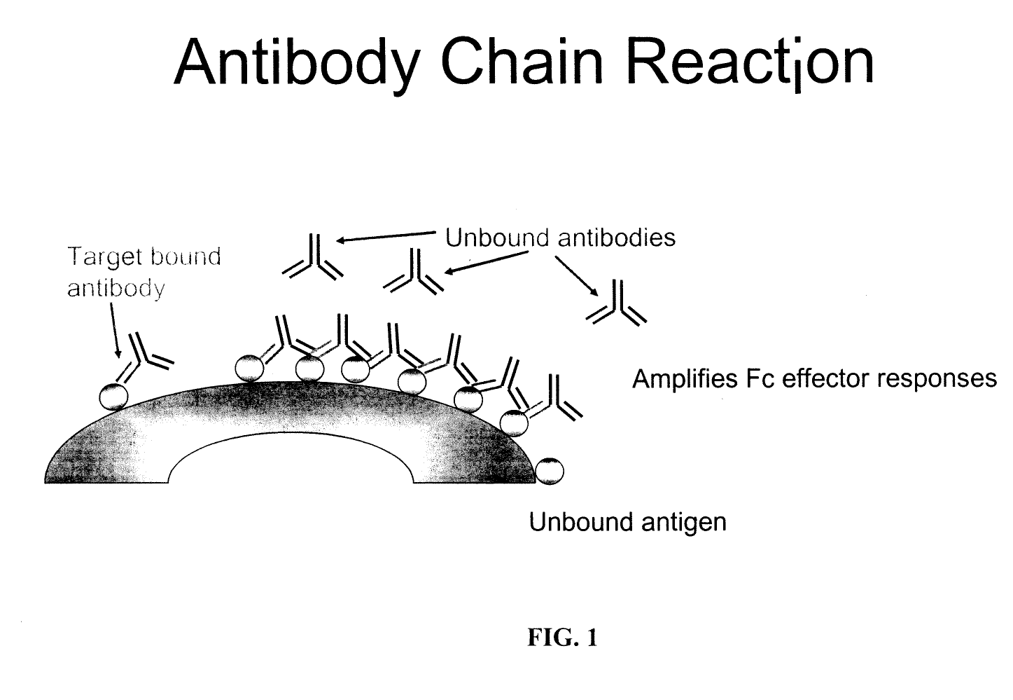

FIG. 2 illustrates the antibody chain reaction by using pairs of antigen-specific and framework specific antibodies (first panel), or bispecific antibodies having both antigen specificity and framework specificity (second panel).

FIG. 3 illustrates the antibody chain reaction by using pairs of antigen-specific and anti-idiotype antibodies (first panel), or bispecific antibodies having both antigen specificity and anti-Fv specificity (second panel).

FIG. 4 illustrates the antibody chain reaction by using pairs of antigen specific antibodies and antibodies specifically binding a complex formed between the first antibody and the target antigen (first panel), or bispecific antibodies having both antigen and anti-complex specificities (second panel).

FIG. 5 illustrates an anti-complex chain reaction, using target specific antibodies and multi-specific antibodies having one specificity that recognizes the target specific antibody-target complex (first panel), or using multi-specific antibodies, specifically recognizing and binding to a target and having an additional specificity recognizing a complex formed between the multi-specific antibody and the target. In each case, the antibody chain formed displays an additional binding region for an additional target.

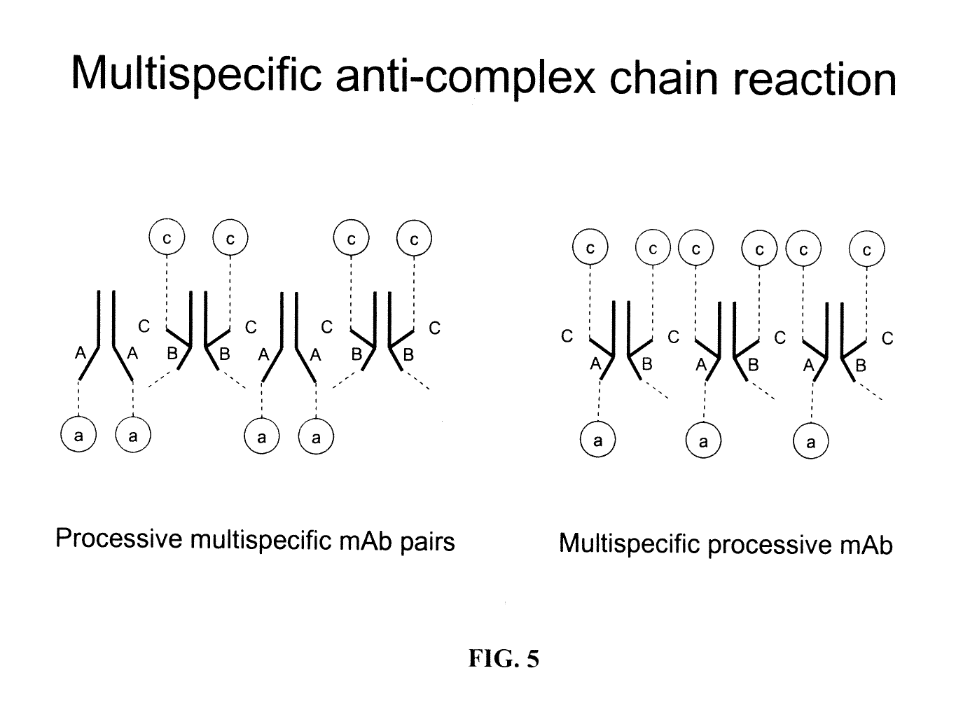

FIG. 6 shows the human VpreB1 sequence of SEQ ID NO: 1; the mouse VpreB2 sequences of SEQ ID NOs: 2 and 3; the human VpreB3 sequence of SEQ ID NO: 4; the human λ5 sequence of SEQ ID NO: 5; and the human λ5-like protein sequence of SEQ ID NO: 6.

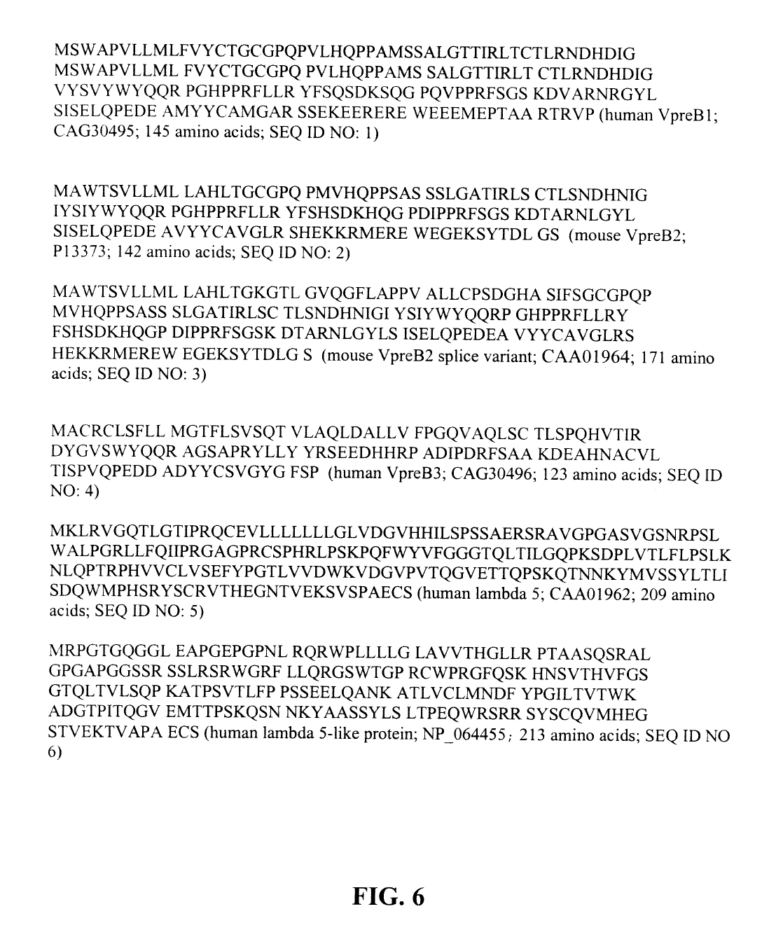

### DETAILED DESCRIPTION OF THE INVENTION

### A. Definitions

Unless defined otherwise, technical and scientific terms used herein have the same meaning as commonly understood by one of ordinary skill in the art to which this invention belongs. Singleton et al., Dictionary of Microbiology and Molecular Biology 2nd ed., J. Wiley & Sons (New York, N.Y. 1994), provides one skilled in the art with a general guide to many of the terms used in the present application.

One skilled in the art will recognize many methods and materials similar or equivalent to those described herein, which could be used in the practice of the present invention. Indeed, the present invention is in no way limited to the methods and materials described. For purposes of the present invention, the following terms are defined below.

The term “oligomer” is used herein in the broadest sense and refers to a substance composed of or comprising multiple, identical or different, repetitive units (repeats), regardless of the number of repetitive units or the molecular weight of the oligomer formed. Thus, the term “oligomer”, as used herein, specifically includes molecules comprising at least two repetitive units and polymers having higher molecular weights and more repetitive units. The oligomeric polypeptide herein might comprise the same or two or more different repeats, which build up the oligomeric polypeptide in a regular repetitive arrangement. In a preferred embodiment, the oligomer will comprise at least 3 or at least 4 or at least 10 repetitive units.

The term “binding molecule” is used in the broadest sense and includes all molecules that show a specific binding affinity for a target. The term specifically includes, without limitation, antibodies and antibody fragments, immunoadhesins, constructs comprising antibody surrogate light chain sequences, binding polypeptides, peptides, and non-peptide small molecules. Constructs comprising antibody surrogate light chain sequences are described in co-pending application Ser. No. 60/920,568, filed on Mar. 27, 2007, the entire disclosure of which is hereby expressly incorporated by reference, and discussed below. Binding polypeptides, other than antibodies and antibody-like molecules, include receptors (binding to ligands), ligands (binding to receptors), and enzymes (binding to a substrate), for example.

The term “bispecific” as used herein refers to at least two binding regions that bind to different targets. Thus, “bispecific” molecules, such as “bispecific” polypeptides are defined herein as having the ability to bind to two or more different targets, and thus specifically include molecules with more than two specificities, such as, for example, trispecific and multi-specific molecules, e.g. antibodies.

The term “bivalent” as used herein refers to at least two binding regions for the same target. Thus, “bivalent” molecules, such as “bivalent” polypeptides are defined herein as having the ability to bind to two or more molecules of the same target. Accordingly, for example, “bivalent” antibodies can crosslink two of the same antigen molecules.

The term “binding region” is used herein in the broadest sense and refers to any region of a polypeptide which is responsible for or participates in selective binding to a target. The term “binding region,” as defined herein includes, for example, all or part of an antibody heavy chain and/or light chain including variable region sequences required for binding, which may be present as part of an antibody fragment or a full length antibody, such as an IgG (e.g., an IgG1, IgG2, IgG3, or IgG4 subtype), IgA1, IgA2, IgD, IgE, or IgM antibody. In terms of antibody-like molecules, the term includes all or part of a receptor binding domain, a ligand binding domain or an enzymatic domain of an antibody-like molecule, e.g. an immunoadhesin. In a specific embodiment, the “binding region” includes the antigen binding region formed by antibody heavy and light chain variable domain sequences. In another embodiment, the “binding region” includes the ligand or receptor binding sequences of a bispecific immunoadhesin molecule, in which one arm of the immunoadhesin molecule can, for example, be a fusion of a receptor- or ligand-binding sequence and an antibody heavy chain constant region sequence, while the other arm of the immunoadhesin molecule may bind a complex formed between such bispecific immunoadhesin molecule and a ligand and receptor, respectively. In yet another embodiment, the “binding region” includes the target-binding sequences from a surrogate light chain, or a construct comprising a surrogate light chain.

The term “bispecific polypeptide” is used herein in the broadest sense and includes all bispecific molecules that comprise a polypeptide sequence. Accordingly, the term “bispecific polypeptide” includes molecules comprising two or more polypeptide chains, which may be connected to each other by non-polypeptide sequences or may be non-covalently associated to each other. Without limitation, the term “bispecific polypeptide” includes bispecific antibodies, bispecific antibody fragments, bispecific immunoadhesins, bispecific molecules comprising surrogate light chain sequences, bispecific T cell receptor (TCR) molecules, and the like. The TCR molecules may be obtained either from T cell clones or hybridomas or as purified TCR preparations.

As used herein, the terms “peptide,” “polypeptide” and “protein” all refer to a primary sequence of amino acids that are joined by covalent “peptide linkages.” In general, a peptide consists of a few amino acids, typically from about 2 to about 50 amino acids, and is shorter than a protein. The term “polypeptide,” as defined herein, encompasses peptides and proteins.

The term “target” is used herein in the broadest sense and refers to any molecule of interest to which a binding molecule binds. The term includes, without limitation, an antigen, a ligand, a receptor, a substrate for an enzyme, or a complex formed between another target and a binding molecule, e.g. a binding polypeptide. Thus, targets specifically include immune complexes formed between an antibody and an antigen.

By “(bispecific) polypeptide-target complex” or “target-(bispecific) polypeptide complex” is meant the association of a polypeptide (e.g. a bispecific polypeptide) and a target such that the polypeptide and the target join each other in a specific, detectable manner. Such complexes include, for example, the association of an antibody and an antigen, ligand and receptor, enzyme and substrate, antibody and anti-idiotype antibody, liganded antibody and antibody binding thereto.

In the context of the present invention, the term “antibody” (Ab) is used in the broadest sense and refers to polypeptides which exhibit binding specificity to a specific antigen, including, without limitation, native and variant monoclonal antibodies and fragments thereof.

“Native antibodies” are usually heterotetrameric glycoproteins of about 150,000 daltons, composed of two identical light (L) chains and two identical heavy (H) chains. Each light chain is linked to a heavy chain by covalent disulfide bond(s), while the number of disulfide linkages varies between the heavy chains of different immunoglobulin isotypes. Each heavy and light chain also has regularly spaced intrachain disulfide bridges. Each heavy chain has, at one end, a variable domain (V_{H}) followed by a number of constant domains. Each light chain has a variable domain at one end (V_{L}) and a constant domain at its other end; the constant domain of the light chain is aligned with the first constant domain of the heavy chain, and the light chain variable domain is aligned with the variable domain of the heavy chain. Particular amino acid residues are believed to form an interface between the light- and heavy-chain variable domains, Chothia et al., J. Mol. Biol. 186:651 (1985); Novotny and Haber, Proc. Natl. Acad. Sci. U.S.A. 82:4592 (1985).

The term “variable” with reference to antibody chains is used to refer to portions of the antibody chains which differ extensively in sequence among antibodies and participate in the binding and specificity of each particular antibody for its particular antigen. Such variability is concentrated in three segments called hypervariable regions both in the light chain and the heavy chain variable domains. The more highly conserved portions of variable domains are called the framework region (FR). The variable domains of native heavy and light chains each comprise four FRs (FR1, FR2, FR3 and FR4, respectively), largely adopting a β-sheet configuration, connected by three hypervariable regions, which form loops connecting, and in some cases forming part of, the β-sheet structure. The hypervariable regions in each chain are held together in close proximity by the FRs and, with the hypervariable regions from the other chain, contribute to the formation of the antigen-binding site of antibodies (see Kabat et al., Sequences of Proteins of Immunological Interest, 5th Ed. Public Health Service, National Institutes of Health, Bethesda, Md. (1991), pages 647-669). The constant domains are not involved directly in binding an antibody to an antigen, but exhibit various effector functions, such as participation of the antibody in antibody-dependent cellular toxicity.

The term “hypervariable region” when used herein refers to the amino acid residues of an antibody which are responsible for antigen-binding. The hypervariable region comprises amino acid residues from a “complementarity determining region” or “CDR” (i.e., residues 30-36 (L1), 46-55 (L2) and 86-96 (L3) in the light chain variable domain and 30-35 (H1), 47-58 (H2) and 93-101 (H3) in the heavy chain variable domain; MacCallum et al., J Mol. Biol. 1996.

The term “framework region” refers to the art recognized portions of an antibody variable region that exist between the more divergent CDR regions. Such framework regions are typically referred to as frameworks 1 through 4 (FR1, FR2, FR3, and FR4) and provide a scaffold for holding, in three-dimensional space, the three CDRs found in a heavy or light chain antibody variable region, such that the CDRs can form an antigen-binding surface.

Depending on the amino acid sequence of the constant domain of their heavy chains, antibodies can be assigned to different classes. There are five major classes of antibodies IgA, IgD, IgE, IgG, and IgM, and several of these may be further divided into subclasses (isotypes), e.g., IgG1, IgG2, IgG3, IgG4, IgA, and IgA2.

The heavy-chain constant domains that correspond to the different classes of immunoglobulins are called α, δ, ε, γ, and μ, respectively.

The “light chains” of antibodies from any vertebrate species can be assigned to one of two clearly distinct types, called kappa (κ) and lambda (λ), based on the amino acid sequences of their constant domains.

The term “monoclonal antibody” is used to refer to an antibody molecule synthesized by a single clone of B cells. The modifier “monoclonal” indicates the character of the antibody as being obtained from a substantially homogeneous population of antibodies, and is not to be construed as requiring production of the antibody by any particular method. Thus, monoclonal antibodies may be made by the hybridoma method first described by Kohler and Milstein, Nature 256:495 (1975); Eur. J. Immunol. 6:511 (1976), by recombinant DNA techniques, or may also be isolated from phage or other antibody libraries.

The term “polyclonal antibody” is used to refer to a population of antibody molecules synthesized by a population of B cells.

“Antibody fragments” comprise a portion of a full length antibody, generally the antigen binding domain(s) or variable domain(s) thereof. Examples of antibody fragments include, but are not limited to, Fab, Fab′, F(ab′)₂, scFv, (scFv)₂, dAb, and complementarity determining region (CDR) fragments, linear antibodies, single-chain antibody molecules, minibodies, diabodies, multispecific antibodies formed from antibody fragments, and, in general, polypeptides that contain at least a portion of an immunoglobulin that is sufficient to confer specific antigen binding to the polypeptide. Specifically within the scope of the invention are bispecific antibody fragments.

The terms “bispecific antibody” and “bispecific antibody fragment” are used herein to refer to antibodies or antibody fragments with binding specificity for at least two targets. If desired, multi-specificity can be combined by multi-valency in order to produce multivalent bispecific antibodies that possess more than one binding site for each of their targets. For example, by dimerizing two scFv fusions via the helix-turn-helix motif, (scFv)₁-hinge-helix-turn-helix-(scFv)₂, a tetravalent bispecific miniantibody was produced (Müller et al., FEBS Lett. 432(1-2):45-9 (1998)). The so-called ‘di-bi-miniantibody’ possesses two binding sites to each of it target antigens.

As used herein, the term “immunoadhesin” designates antibody-like molecules which combine the “binding domain” of a heterologous protein (an “adhesin”, e.g. a receptor, ligand or enzyme) with the effector functions of immunoglobulin constant domains. Structurally, the immunoadhesins comprise a fusion of the adhesin amino acid sequence with the desired binding specificity which is other than the antigen recognition and binding site (antigen combining site) of an antibody (i.e. is “heterologous”) and an immunoglobulin constant domain sequence. The immunoglobulin constant domain sequence in the immunoadhesin may be obtained from any immunoglobulin, such as IgG1, IgG.2, IgG3, or IgG4 subtypes, IgA, IgE, IgD or IgM. For further details of immunoadhesins, ligand binding domains and receptor binding domains see, e.g. U.S. Pat. Nos. 5,116,964; 5,714,147; and 6,406,604, the disclosures of which are hereby expressly incorporated by reference.

The term “ligand binding domain” as used herein refers to any native cell-surface receptor or any region or derivative thereof retaining at least a qualitative ligand binding ability, and preferably the biological activity of a corresponding native receptor. In a specific embodiment, the receptor is from a cell-surface polypeptide having an extracellular domain which is homologous to a member of the immunoglobulin supergenefamily. Other typical receptors, are not members of the immunoglobulin supergenefamily but are nonetheless covered by this definition, are receptors for cytokines, and receptor tyrosine kinases, cell adhesion molecules, and the like.

The term “receptor binding domain” is used to designate any native ligand for a receptor, or any region or derivative of such native ligand retaining at least a qualitative receptor binding ability, and preferably the biological activity of a corresponding native ligand. This definition, among others, includes binding sequences from ligands for the above-mentioned receptors.

The term “epitope” as used herein, refers to a sequence of at least about 3 to 5, preferably at least about 5 to 10, or at least about 5 to 15 amino acids, and typically not more than about 500, or about 1,000 amino acids, which define a sequence that by itself, or as part of a larger sequence, binds to an antibody generated in response to such sequence. An epitope is not limited to a polypeptide having a sequence identical to the portion of the parent protein from which it is derived. Indeed, viral genomes are in a state of constant change and exhibit relatively high degrees of variability between isolates. Thus the term “epitope” encompasses sequences identical to the native sequence, as well as modifications, such as deletions, substitutions and/or insertions to the native sequence. Generally, such modifications are conservative in nature but non-conservative modifications are also contemplated. The term includes “mimotopes,” i.e. sequences that do not identify a continuous linear native sequence or do not necessarily occur in a native protein, but functionally mimic an epitope on a native protein. The term “epitope” includes linear and conformational epitopes.

As used herein, the term “conformational epitope” refers to an epitope formed by discontinuous portions of a protein having structural features of corresponding sequences in the properly folded full-length native protein. The length of the epitope-defining sequence (the sequence including the discontinuous portions making up the conformational epitope) can greatly vary as these epitopes are formed by the three-dimensional structure of the protein. Thus, amino acids defining the epitope can be relatively few in number, widely dispersed along the length of the molecule, being brought into correct epitope conformation via folding. The portions of the protein between the residues defining the epitope may not be critical to the conformational structure of the epitope. For example, deletion or substitution of these intervening sequences may not affect the conformational epitope provided that the sequences critical to epitope conformation are maintained. Thus, a “conformational epitope,” as defined herein, is not required to be identical to a native conformational epitope, but rather includes conformationally constrained structures that regenerate (exhibit) essential properties (such as qualitative antibody-binding properties) of native conformational epitopes.

“Linear epitopes” are fragments of discontinuous or conformational epitopes.

Regions of a given polypeptide that include an epitope can be identified using any number of epitope mapping techniques, well known in the art. See, e.g., Epitope Mapping Protocols in Methods in Molecular Biology, Vol. 66 (Glenn E. Morris, Ed., 1996) Humana Press, Totowa, N.J.

The term “surrogate light chain,” as used herein, refers to a dimer formed by the non-covalent association of a VpreB and a λ5 protein.

The term “VpreB” is used herein in the broadest sense and refers to any native sequence or variant VpreB polypeptide, including, without limitation, human VpreB1 of SEQ ID NO: 1, mouse VpreB2 of SEQ ID NOS: 2 and 3, human VpreB3 of SEQ ID NO: 4 and isoforms, including splice variants and variants formed by posttranslational modifications, other mammalian homologues thereof, as well as variants of such native sequence polypeptides.

The term “λ5” is used herein in the broadest sense and refers to any native sequence or variant λ5 polypeptide, including, without limitation, human λ5 of SEQ ID NO: 5, human λ5-like protein of SEQ ID NO: 6, and their isoforms, including splice variants and variants formed by posttranslational modifications, other mammalian homologous thereof, as well a variants of such native sequence polypeptides.

The terms “variant VpreB polypeptide” and “a variant of a VpreB polypeptide” are used interchangeably, and are defined herein as a polypeptide differing from a native sequence VpreB polypeptide at one or more amino acid positions as a result of an amino acid modification. The “variant VpreB polypeptide,” as defined herein, will be different from a native antibody λ or κ light chain sequence, or a fragment thereof. The “variant VpreB polypeptide” will preferably retain at least about 65%, or at least about 70%, or at least about 75%, or at least about 80%, or at least about 85%, or at least about 90%, or at least about 95%, or at least about 98% sequence identity with a native sequence VpreB polypeptide. In another preferred embodiment, the “variant VpreB polypeptide” will be less then 95%, or less than 90%, or less then 85%, or less than 80%, or less than 75%, or less then 70%, or less than 65%, or less than 60% identical in its amino acid sequence to a native antibody λ or κ light chain sequence.

The terms “variant λ5 polypeptide” and “a variant of a λ5 polypeptide” are used interchangeably, and are defined herein as a polypeptide differing from a native sequence λ5 polypeptide at one or more amino acid positions as a result of an amino acid modification. The “variant λ5 polypeptide,” as defined herein, will be different from a native antibody λ or κ light chain sequence, or a fragment thereof. The “variant λ5 polypeptide” will preferably retain at least about 65%, or at least about 70%, or at least about 75%, or at least about 80%, or at least about 85%, or at least about 90%, or at least about 95%, or at least about 98% sequence identity with a native sequence λ5 polypeptide. In another preferred embodiment, the “variant λ5 polypeptide” will be less then 95%, or less than 90%, or less then 85%, or less than 80%, or less than 75%, or less then 70%, or less than 65%, or less than 60% identical in its amino acid sequence to a native antibody λ or κ light chain sequence.

The term “VpreB sequence” is used herein to refer to the sequence of “VpreB,” as hereinabove defined, or a fragment thereof.

The term “λ5 sequence” is used herein to refers to the sequence of “λ5,” as hereinabove defined, or a fragment thereof.

The term “surrogate light chain sequence,” as defined herein, means any polypeptide sequence that comprises a “VpreB sequence” and/or a “λ5 sequence,” as hereinabove defined. The “surrogate light chain sequence,” as defined herein, includes, without limitation, the human VpreB1 sequence of SEQ ID NO 1, the mouse VpreB2 sequences of SEQ ID NOS: 2 and 3, and the human VpreB3 sequence of SEQ ID NO: 4, and their various isoforms, including splice variants and variants formed by posttranslational modifications, homologues thereof in other mammalian species, as well as fragments and variants thereof. The term “surrogate light chain sequence” additionally includes, without limitation, the human λ5 sequence of SEQ ID NO: 5, the human λ5-like sequence of SEQ ID NO: 6, and their isoforms, including splice variants and variants formed by posttranslational modifications, homologues thereof in other mammalian species, as well as fragments and variants thereof. The term “surrogate light chain sequence” additionally includes a sequence comprising both VpreB and λ5 sequences as hereinabove defined.

The “surrogate light chain sequence” may be optionally conjugated to a heterogeneous amino acid sequence, or any other heterogeneous component, to form a “surrogate light chain construct” herein. Thus, the term, “surrogate light chain construct” is used in the broadest sense and includes any and all additional heterogeneous components, including a heterogeneous amino acid sequence, nucleic acid, and other molecules conjugated to a surrogate light chain sequence, wherein “conjugation” is defined below.

In the context of the polypeptides of the present invention, the term “heterogeneous amino acid sequence,” relative to a first amino acid sequence, is used to refer to an amino acid sequence not naturally associated with the first amino acid sequence, at least not in the form it is present in the surrogate light chain constructs herein. Thus, a “heterogenous amino acid sequence” relative to a VpreB is any amino acid sequence not associated with native VpreB in its native environment, including, without limitation, λ5 sequences that are different from those λ5 sequences that, together with VpreB, form the surrogate light chain On developing B cells, such as amino acid sequence variants, e.g. truncated and/or derivatized λ5 sequences. A “heterogeneous amino acid sequence” relative to a VpreB also includes λ5 sequences covalently associated with, e.g. fused to, VpreB, including native sequence λ5, since in their native environment, the VpreB and λ5 sequences are not covalently associated, e.g. fused, to each other.

The terms “conjugate,” “conjugated,” and “conjugation” refer to any and all forms of covalent or non-covalent linkage, and include, without limitation, direct genetic or chemical fusion, coupling through a linker or a cross-linking agent, and non-covalent association, for example through Van der Waals forces, or by using a leucine zipper.

The term “fusion” is used herein to refer to the combination of amino acid sequences of different origin in one polypeptide chain by in-frame combination of their coding nucleotide sequences. The term “fusion” explicitly encompasses internal fusions, i.e., insertion of sequences of different origin within a polypeptide chain, in addition to fusion to one of its termini.

The term “amino acid” or “amino acid residue” typically refers to an amino acid having its art recognized definition such as an amino acid selected from the group consisting of: alanine (Ala); arginine (Arg); asparagine (Asn); aspartic acid (Asp); cysteine (Cys); glutamine (Gln); glutamic acid (Glu); glycine (Gly); histidine (His); isoleucine (Ile): leucine (Leu); lysine (Lys); methionine (Met); phenylalanine (Phe); proline (Pro); serine (Ser); threonine (Thr); tryptophan (Trp); tyrosine (Tyr); and valine (Val) although modified, synthetic, or rare amino acids may be used as desired. Thus, modified and unusual amino acids listed in 37 CFR 1.822(b)(4) are included within this definition and expressly incorporated herein by reference. Amino acids can be subdivided into various sub-groups. Thus, amino acids can be grouped as having a nonpolar side chain (e.g., Ala, Cys, Ile, Leu, Met, Phe, Pro, Val); a negatively charged side chain (e.g., Asp, Glu); a positively charged side chain (e.g., Arg, His, Lys); or an uncharged polar side chain (e.g., Asn, Cys, Gln, Gly, His, Met, Phe, Ser, Thr, Trp, and Tyr). Amino acids can also be grouped as small amino acids (Gly, Ala), nucleophilic amino acids (Ser, His, Thr, Cys), hydrophobic amino acids (Val, Leu, Ile, Met, Pro), aromatic amino acids (Phe, Tyr, Trp, Asp, Glu), amides (Asp, Glu), and basic amino acids (Lys, Arg).

The term “variant” with respect to a reference polypeptide refers to a polypeptide that possesses at least one amino acid mutation or modification (i.e., alteration) as compared to a native polypeptide. Variants generated by “amino acid modifications” can be produced, for example, by substituting, deleting, inserting and/or chemically modifying at least one amino acid in the native amino acid sequence.

An “amino acid modification” refers to a change in the amino acid sequence of a predetermined amino acid sequence. Exemplary modifications include an amino acid substitution, insertion and/or deletion.

An “amino acid modification at” a specified position, refers to the substitution or deletion of the specified residue, or the insertion of at least one amino acid residue adjacent the specified residue. By insertion “adjacent” a specified residue is meant insertion within one to two residues thereof. The insertion may be N-terminal or C-terminal to the specified residue.

An “amino acid substitution” refers to the replacement of at least one existing amino acid residue in a predetermined amino acid sequence with another different “replacement” amino acid residue. The replacement residue or residues may be “naturally occurring amino acid residues” (i.e. encoded by the genetic code) and selected from the group consisting of: alanine (Ala); arginine (Arg); asparagine (Asn); aspartic acid (Asp); cysteine (Cys); glutamine (Gln); glutamic acid (Glu); glycine (Gly); histidine (His); isoleucine (Ile): leucine (Leu); lysine (Lys); methionine (Met); phenylalanine (Phe); proline (Pro); serine (Ser); threonine (Thr); tryptophan (Trp); tyrosine (Tyr); and valine (Val). Substitution with one or more non-naturally occurring amino acid residues is also encompassed by the definition of an amino acid substitution herein.

A “non-naturally occurring amino acid residue” refers to a residue, other than those naturally occurring amino acid residues listed above, which is able to covalently bind adjacent amino acid residues(s) in a polypeptide chain. Examples of non-naturally occurring amino acid residues include norleucine, ornithine, norvaline, homoserine and other amino acid residue analogues such as those described in Ellman et al. Meth. Enzym. 202:301 336 (1991). To generate such non-naturally occurring amino acid residues, the procedures of Noren et al. Science 244:182 (1989) and Ellman et al., supra, can be used. Briefly, these procedures involve chemically activating a suppressor tRNA with a non-naturally occurring amino acid residue followed by in vitro transcription and translation of the RNA.

An “amino acid insertion” refers to the incorporation of at least one amino acid into a predetermined amino acid sequence. While the insertion will usually consist of the insertion of one or two amino acid residues, the present application contemplates larger “peptide insertions”, e.g. insertion of about three to about five or even up to about ten amino acid residues. The inserted residue(s) may be naturally occurring or non-naturally occurring as disclosed above.

An “amino acid deletion” refers to the removal of at least one amino acid residue from a predetermined amino acid sequence.

The term “polynucleotide(s)” refers to nucleic acids such as DNA molecules and RNA molecules and analogs thereof (e.g., DNA or RNA generated using nucleotide analogs or using nucleic acid chemistry). As desired, the polynucleotides may be made synthetically, e.g., using art-recognized nucleic acid chemistry or enzymatically using, e.g., a polymerase, and, if desired, be modified. Typical modifications include methylation, biotinylation, and other art-known modifications. In addition, the nucleic acid molecule can be single-stranded or double-stranded and, where desired, linked to a detectable moiety.

The term “mutagenesis” refers to, unless otherwise specified, any art recognized technique for altering a polynucleotide or polypeptide sequence. Preferred types of mutagenesis include error prone PCR mutagenesis, saturation mutagenesis, or other site directed mutagenesis.

“Site-directed mutagenesis” is a technique standard in the art, and is conducted using a synthetic oligonucleotide primer complementary to a single-stranded phage DNA to be mutagenized except for limited mismatching, representing the desired mutation. Briefly, the synthetic oligonucleotide is used as a primer to direct synthesis of a strand complementary to the single-stranded phage DNA, and the resulting double-stranded DNA is transformed into a phage-supporting host bacterium. Cultures of the transformed bacteria are plated in top agar, permitting plaque formation from single cells that harbor the phage. Theoretically, 50% of the new plaques will contain the phage having, as a single strand, the mutated form; 50% will have the original sequence. Plaques of interest are selected by hybridizing with kinased synthetic primer at a temperature that permits hybridization of an exact match, but at which the mismatches with the original strand are sufficient to prevent hybridization. Plaques that hybridize with the probe are then selected, sequenced and cultured, and the DNA is recovered.

The term “vector” is used to refer to a rDNA molecule capable of autonomous replication in a cell and to which a DNA segment, e.g., gene or polynucleotide, can be operatively linked so as to bring about replication of the attached segment. Vectors capable of directing the expression of genes encoding for one or more polypeptides are referred to herein as “expression vectors.” The term “control sequences” refers to DNA sequences necessary for the expression of an operably linked coding sequence in a particular host organism. The control sequences that are suitable for prokaryotes, for example, include a promoter, optionally an operator sequence, and a ribosome binding site. Eukaryotic cells are known to utilize promoters, polyadenylation signals, and enhancers.

Nucleic acid is “operably linked” when it is placed into a functional relationship with another nucleic acid sequence. For example, DNA for a presequence or secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are contiguous, and, in the case of a secretory leader, contiguous and in reading phase. However, enhancers do not have to be contiguous. Linking is accomplished by ligation at convenient restriction sites. If such sites do not exist, the synthetic oligonucleotide adaptors or linkers are used in accordance with conventional practice.

Percent amino acid sequence identity may be determined using the sequence comparison program NCBI-BLAST2 (Altschul et al., Nucleic Acids Res. 25:3389-3402 (1997)). The NCBI-BLAST2 sequence comparison program may be downloaded from http://www.ncbi.nlm.nih.gov or otherwise obtained from the National Institute of Health, Bethesda, Md. NCBI-BLAST2 uses several search parameters, wherein all of those search parameters are set to default values including, for example, unmask=yes, strand=all, expected occurrences=10, minimum low complexity length=15/5, multi-pass e-value=0.01, constant for multi-pass=25, dropoff for final gapped alignment=25 and scoring matrix=BLOSUM62.

Antibody-dependent cell-mediated cytotoxicity” and “ADCC” are used herein to refer to a cell-mediated reaction in which nonspecific cytotoxic cells that express FcRs (e.g. Natural Killer (NK) cells, neutrophils, and macrophages) recognize bound antibody on a target cell and subsequently cause lysis of the target cell. Various immune cells express different Fc receptors (FcRs). Thus, the primary cells for mediating ADCC, NK cells, express FcγRIII only, whereas monocytes express FcγRI, FcγRII and FcγRIII.

### B. Detailed Description

Techniques for performing the methods of the present invention are well known in the art and described in standard laboratory textbooks, including, for example, Ausubel et al., Current Protocols of Molecular Biology, John Wiley and Sons (1997); Molecular Cloning: A Laboratory Manual, Third Edition, J. Sambrook and D. W. Russell, eds., Cold Spring Harbor, N.Y., USA, Cold Spring Harbor Laboratory Press, 2001; O'Brian et al., Analytical Chemistry of Bacillus Thuringiensis, Hickle and Fitch, eds., Am. Chem. Soc., 1990; Bacillus thuringiensis: biology, ecology and safety, T. R. Glare and M. O'Callaghan, eds., John Wiley, 2000; Antibody Phage Display, Methods and Protocols, Humana Press, 2001; and Antibodies, G. Subramanian, ed., Kluwer Academic, 2004. Mutagenesis can, for example, be performed using site-directed mutagenesis (Kunkel et al., Proc. Natl. Acad. Sci. USA 82:488-492 (1985)). PCR amplification methods are described in U.S. Pat. Nos. 4,683,192, 4,683,202, 4,800,159, and 4,965,188, and in several textbooks including “PCR Technology: Principles and Applications for DNA Amplification”, H. Erlich, ed., Stockton Press, New York (1989); and PCR Protocols: A Guide to Methods and Applications, Innis et al., eds., Academic Press, San Diego, Calif. (1990).

Antibody Chain Reaction

The present invention concerns a new method for cross-linking identical or different binding molecules to create oligomers comprising repeat units of the binding molecules linked and having binding specificity for a desired target. Since the method is illustrated by way of cross-linking antibodies, before or after binding to a target antigen, it is referred to as “antibody chain reaction.” It should be understood, however, that the method is not limited to antibodies, and the approaches illustrated by reference to antibodies can be extended to other binding molecules, such as binding polypeptides, following the teaching of the present invention and general knowledge in the relevant art.

a. Anti-Framework Chain Reaction

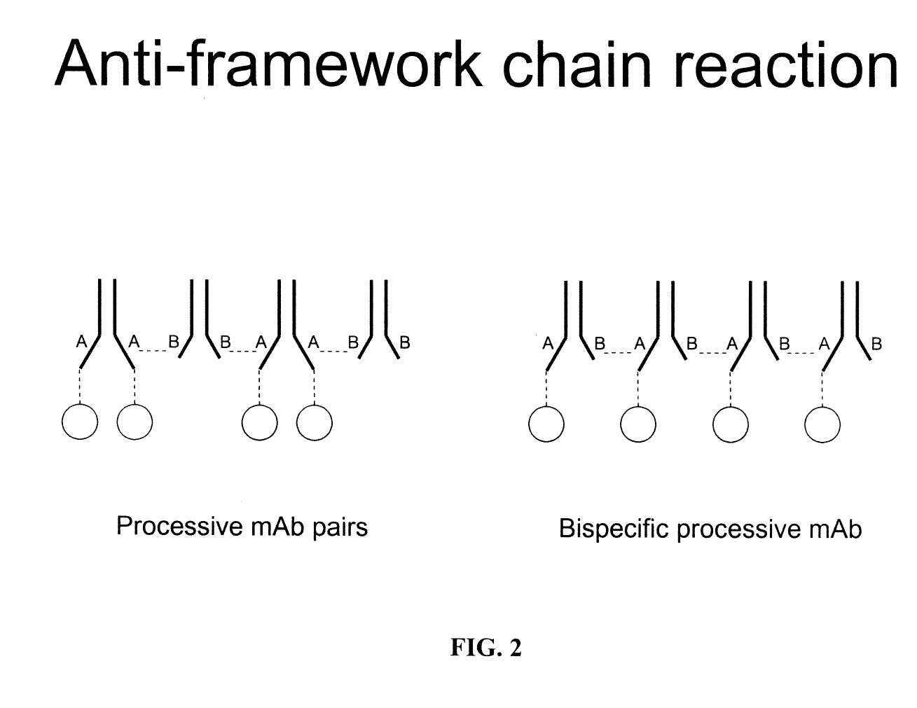

In one embodiment, the chain reaction is between two different antibodies or antibody fragments, where one of the antibodies binds to a target antigen while the other antibody binds to an epitope within the framework region of the first antibody (see, FIG. 2, first panel). As a result, when the two antibodies are incubated under appropriate conditions, the anti-framework antibodies will cross-link the antigen specific antibodies, and an antibody chain, composed of alternating repeat units of the antigen specific antibodies and the anti-framework antibodies will form. Contacting can take place in the presence or absence of the target antigen. When the target antigen is present, the antigen-specific antibodies will both bind the target antigen and become cross-linked by the anti-framework antibodies. Alternatively, the antibody chain reaction can take place in the absence of the target antigen, and the oligomer composed of alternating repeat units of the two types of antibodies can be subsequently contacted with and bind to the antigen.

In another version of this embodiment, the chain reaction results from linking bispecific antibodies or antibody fragments, where one arm of the bispecific antibody or antibody fragment binds to a target antigen and the other arm binds to the framework on another molecule of the same bispecific antibody (see, FIG. 2, second panel). Just as before, cross-linking can take place in the presence or absence of the antigen, and the oligomer, composed on repeat units of identical bispecific antibodies, binds to multiple molecules of the target antigen.

This approach, briefly referred to as “anti-framework chain reaction” is expected to create a highly avid and potentially functionally non-dissociative interaction with the target antigen, thereby increasing clearance of the target antigen. Thus, this approach holds benefits in any field where immune clearance is needed or beneficial. For example, the oligomers produced by the anti-framework chain reaction can be useful in facilitating the clearance of http:/www.rcsb.org/pdb/cgi/explore.cgi?pid=49751117326028&page=0&pdbId=1EO8 infectious pathogens, such as viruses, including those representing a major threat for global public health, e.g. influenza viruses, hepatitis A, B, C viruses, and HIV. As a result, the cross-linked antibodies provide benefit, for example, in the treatment of infectious diseases including, without limitation, viral diseases, such as human immunodeficiency virus (HIV), hepatitis A, B, and C virus, herpes simplex virus (HSV), cytomegalovirus (CMV), Epstein-Barr virus (EBV), or human papilloma virus (HPV) infections; diseases caused by parasites, such as Plasmodia species, Leishmania species, e.g. Leishmania major, Schistosoma species, Trypanosoma species; and bacterial infections, such as infections caused by Mycobacteria, in particular, M. tuberculosis, M. leprae, Yersinia pseudotuberculosis, Salmonella typhimurium, Listeria Monocytogenes, Streptococci, E. coli, Staphylococci; fungal infections, such as those caused by Candida species, Aspergillus species, and Pneumocystis carinii.

b. Anti-Fv Chain Reaction

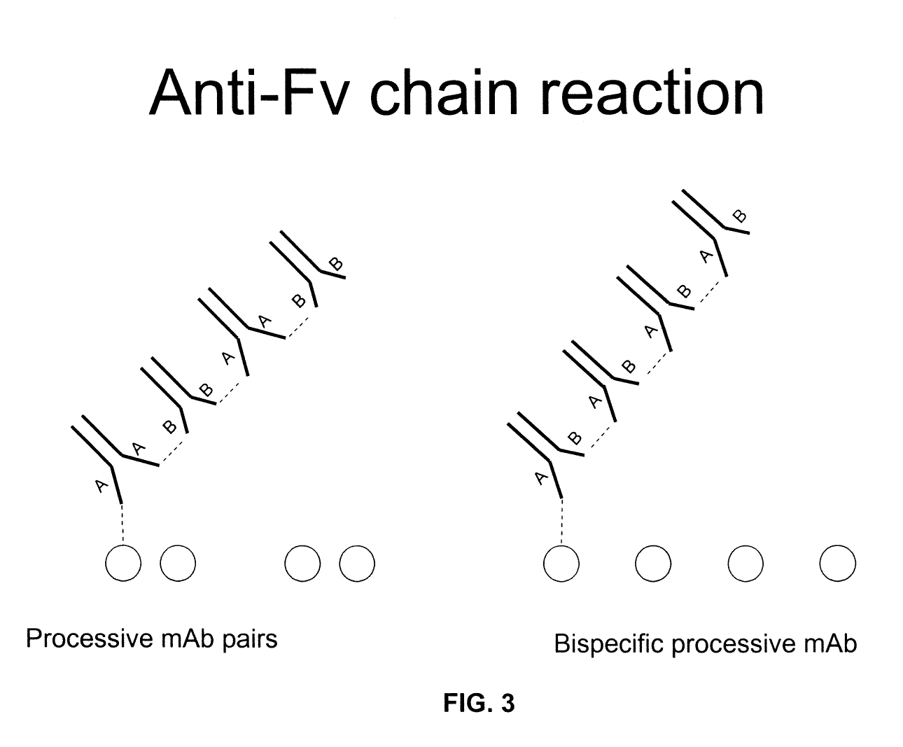

In this embodiment, the chain reaction is between two different antibodies or antibody fragments, where one of the antibodies binds to a target antigen while the other antibody binds to an epitope within the Fv (variable) region of the first antibody (see, FIG. 3, first panel). As a result, when the two antibodies are incubated under appropriate conditions, the anti-Fv antibodies will cross-link the antigen specific antibodies, and an oligomer, composed of alternating repeat units of the antigen specific antibodies and the anti-Fv antibodies will form. Contacting can take place in the presence or absence of the target antigen. Since cross-linking of the antibodies occurs through the Fv (variable) region of the first antibody, the oligomer formed by the chain reaction will bind to the antigen at one site, or a few sites, only, and will emerge from the surface, providing a protruding and repetitive Fc presenting structure.

When the target antigen is present, the antigen-specific antibodies will both bind the target antigen and become cross-linked by the anti-framework antibodies. Alternatively, the antibody chain reaction can take place in the absence of the target antigen, and the oligomer composed of alternating repeat units of the two types of antibodies can be subsequently contacted with and bind to the antigen.

In another version of this embodiment, the chain reaction results from linking bispecific antibodies or antibody fragments, where one arm of the bispecific antibody or antibody fragment binds to a target antigen and the other arm binds to the Fv (variable) region on another molecule of the same bispecific antibody (see, FIG. 3, second panel). Just as before, cross-linking can take place in the presence or absence of the antigen, and the oligomer, composed on repeat units of identical bispecific antibodies, will have one, or just a few, points of contact with the target antigen, and protrudes away from the target antigen.

The emerging structure resulting from this type of chain reaction can be viewed as an “opsonizing” beacon for immune clearance, and can be used whenever enhancement of immune clearance is beneficial, including, for example, clearance of various pathogens, and prevention and treatment of the associated diseases, as discussed above. Since the immunogenic binding site (idiotype) of the anti-idiotype antibodies mimics the antigen, the anti-idiotype antibodies, as single agents or in complexed form, are expected to generate a strong immune response to tumor antigens, and be effective in the prevention and treatment of a variety of cancers, including B neoplasms, including B cell lymphomas, such as non-Hodgkin's lymphoma (NHL); follicular center cell (FCC) lymphomas; acute lymphocytic leukemia (ALL); chronic lymphocytic leukemia (CLL); and Hairy cell leukemia. The non-Hodgkins lymphoma include low grade/follicular non-Hodgkin's lymphoma (NHL), small lymphocytic (SL) NHL, intermediate grade/follicular NHL, intermediate grade diffuse NHL, high grade immunoblastic NHL, high grade lymphoblastic NHL, high grade small non-cleaved cell NHL, bulky disease NHL, plasmacytoid lymphocytic lymphoma, mantle cell lymphoma, AIDS-related lymphoma and Waldenstrom's macroglobulinemia, for example. This approach can be combined with other therapeutic approaches for the treatment of the target disease or condition, including, for example, other therapies for the treatment of B cell lymphomas, including administration of RITUXAN® (rituximab), and other antibodies to B cell antigens, such as CD20 or CD22.

(c) Anti-Complex Chain Reaction

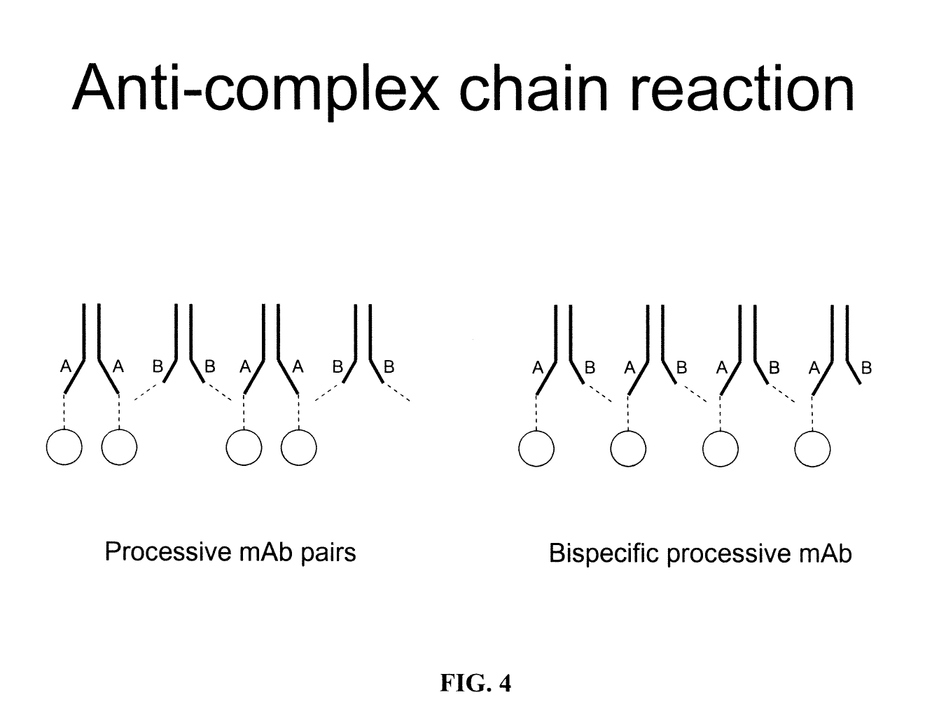

In this embodiment, the chain reaction is between two different antibodies or antibody fragments, where one of the antibodies binds to a target antigen while the other antibody binds to a complex between the antibody and the target antigen (see, FIG. 4, first panel). As a result, when the two antibodies are presented under appropriate conditions, the anti-complex antibodies will cross-link the antigen specific antibodies, and an antibody chain, composed of alternating repeat units of the antigen specific antibodies and the anti-complex antibodies will form. Processive contact occurs when the target antigen is present. The chain reaction takes place as a result of the antigen-specific antibodies both binding the target antigen and cross-linking by the anti-complex antibodies.

In another version of this embodiment, the chain reaction results from linking bispecific antibodies or antibody fragments, where one arm of the bispecific antibody binds to a target antigen and the other arm binds to the complex formed between the bispecific antibody and the target antigen (see, FIG. 4, second panel). Just as before, cross-linking can take place in the presence or absence of the antigen, and the oligomer, composed on repeat units of identical bispecific antibodies, binds to multiple molecules of the target antigen.

This approach can be used to processively crosslink antigen specific antibodies until the antigen becomes exhausted. Thus, this approach provides a target-dependent and self-assembling “opsonizing” net for immune clearance. In addition, the complex-specific oligomerizing monoclonal antibodies hold great promise as a combination cancer therapy with existing therapeutic antibodies, such as, for example, HERCEPTIN® (trastuzumab), or RITUXAN® (rituximab) and/or other anti-cancer agents.

The listed approaches are merely representative. One of ordinary skill will recognize that many further embodiments and variants are possible, using the principle of antibody chain reaction described herein. Furthermore, the methods described herein are not limited to antibodies. All binding molecules, including antibody surrogate light chain constructs, receptors, ligands and enzymes, whether mono-, bi- or multifunctional, are specifically contemplated for use in the chain reactions of the present invention.

Sources and Preparation of Antibodies

In certain embodiments, the methods of the present invention include the use of antibodies, such as antibodies binding to a target antigen. The antigen-specific antibodies herein include all therapeutic antibodies in clinical use or under development, all commercially available antibodies, and antibodies to any antigen. Other antibodies may be prepared by methods well known in the art.

(a) Hybridoma Method

Monoclonal antibodies may be made using the hybridoma method first described by Kohler et al., Nature, 256:495 (1975), or may be made by recombinant DNA methods. In the hybridoma method, a mouse or other appropriate host animal, such as a hamster or macaque monkey, is immunized as hereinabove described to elicit lymphocytes that produce or are capable of producing antibodies that will specifically bind to the protein used for immunization. Alternatively, lymphocytes may be immunized in vitro. Lymphocytes then are fused with myeloma cells using a suitable fusing agent, such as polyethylene glycol, to form a hybridoma cell (Goding, Monoclonal Antibodies: Principles and Practice, pp. 59-103 (Academic Press, 1986)).

The hybridoma cells thus prepared are seeded and grown in a suitable culture medium that preferably contains one or more substances that inhibit the growth or survival of the unfused, parental myeloma cells. For example, if the parental myeloma cells lack the enzyme hypoxanthine guanine phosphoribosyl transferase (HGPRT or HPRT), the culture medium for the hybridomas typically will include hypoxanthine, aminopterin, and thymidine (HAT medium), which substances prevent the growth of HGPRT-deficient cells. Culture medium in which hybridoma cells are growing is assayed for production of monoclonal antibodies directed against the antigen. Preferably, the binding specificity of monoclonal antibodies produced by hybridoma cells is determined by immunoprecipitation or by an in vitro binding assay, such as radioimmunoassay (RIA) or enzyme-linked immunoabsorbent assay (ELISA).

(b) Recombinant Production of Antibodies

For recombinant production of antibodies, the nucleic acid encoding the antibody in question is isolated and inserted into a replicable vector for further cloning (amplification of the DNA) or for expression. DNA encoding the monoclonal antibody is readily isolated and sequenced using conventional procedures (e.g., by using oligonucleotide probes that are capable of binding specifically to genes encoding the heavy and light chains of the antibody). The vector components generally include, but are not limited to, one or more of the following: a signal sequence, an origin of replication, one or more marker genes, an enhancer element, a promoter, and a transcription termination sequence.

Suitable host cells for cloning or expressing the DNA in the vectors herein are the prokaryote, yeast, or higher eukaryote cells described above. Suitable prokaryotes for this purpose include eubacteria, such as Gram-negative or Gram-positive organisms. Preferred E. coli cloning hosts include, for example, E. coli 294 (ATCC 31,446), E. coli B, E. coli X 1776 (ATCC 31,537), and E. coli W3110 (ATCC 27,325).

In addition to prokaryotes, eukaryotic microbes such as filamentous fungi or yeast are suitable cloning or expression hosts for antibody-encoding vectors. Saccharomyces cerevisiae, or common baker's yeast, is the most commonly used among lower eukaryotic host microorganisms. However, a number of other genera, species, and strains are commonly available and useful herein, such as Schizosaccharomyces pombe; Kluyveromyces hosts such as, e.g., K. lactic, K. fragilis (ATCC 12,424), K. bulgaricus (ATCC 16,045), K. wickeramii (ATCC 24,178), K. waltii (ATCC 56,500), K. drosophilarum (ATCC 36,906), K. thermotolerans, and K. marxianus; yarrowia (EP 402,226); Pichia pastoris (EP 183,070); Candida; Trichoderma reesia (EP 244,234); Neurospora crassa; Schwanniomyces such as Schwanniomyces occidentalis; and filamentous fungi such as, e.g., Neurospora, Penicillium, Tolypocladium, and Aspergillus hosts such as A. nidulans and A. niger.

Suitable host cells for the expression of glycosylated antibody are derived from multicellular organisms. Examples of invertebrate cells include plant and insect cells. Numerous baculoviral strains and variants and corresponding permissive insect host cells from hosts such as Spodoptera frugiperda (caterpillar), Aedes aegypti (mosquito), Aedes albopictus (mosquito), Drosophila melanogaster (fruitfly), and Bombyx mori have been identified. A variety of viral strains for transfection are publicly available, e.g., the L-1 variant of Autographa californica NPV and the Bm-5 strain of Bombyx mori NPV, and such viruses may be used as the virus herein according to the present invention, particularly for transfection of Spodoptera frugiperda cells. Plant cell cultures of cotton, corn, potato, soybean, petunia, tomato, and tobacco can also be utilized as hosts.

Interest has been greatest in vertebrate cells, including mammalian cells lines, such as monkey kidney CV1 line transformed bySV40 (COS-7, ATCC CRL 1651); human embryonic kidney line (293 or 293 cells subloned for growth in suspension culture, Graham et al, J. Gen Virol. 36:59 (1977)); baby hamster kidney cells (BHK, ATCC CCL 10); Chinese hamster ovary cells/−DHFR (CHO, Urlaub et al., Proc. Natl. Acad. Sci. USA 77:4216 (1980)); mouse sertoli cells (TM4, Mather, Biol. Reprod. 23:243-251 (1980)); monkey kidney cells (CV1 ATCC CCL 70); African green monkey kidney cells (VERO-76, ATCC CRL-1587); human cervical carcinoma cells (HELA, ATCC CCL 2); canine kidney cells (MDCK, ATCC CCL 34); buffalo rat liver cells (BRL 3A, ATCC CRL 1442); human lung cells (W138, ATCC CCL 75); human liver cells (Hep G2, HB 8065); mouse mammary tumor (MMT 060562, ATCC CCL51); TRI cells (Mather et al., Annals N.Y. Acad. Sci. 383:44-68 (1982)); MRC 5 cells; FS4 cells; and a human hepatoma line (Hep G2).

The host cells may be cultured in a variety of media, which are commercially available an can be used following manufacturers' recommendations and instructions.

(c) Antibody Libraries

In a further embodiment, antibodies or antibody fragments can be isolated from antibody libraries. Antibody libraries are well known in the art. The libraries used to identify antibodies with the desired antigen binding specificity in accordance with the present invention are preferably in the form of a display. Antibodies and antibody fragments have been displayed on the surface of filamentous phage that encode the antibody genes (Hoogenboom and Winter J. Mol. Biol., 222:381 388 (1992); McCafferty et al., Nature 348(6301):552 554 (1990); Griffiths et al. EMBO J., 13(14):3245-3260 (1994)). For a review of techniques for selecting and screening antibody libraries see, e.g., Hoogenboom, Nature Biotechnol. 23(9):1105-1116 (2005). In addition, there are systems known in the art for display of heterologous proteins and fragments thereof, including antibodies and antibody fragments, on the surface of Escherichia coli (Agterberg et al., Gene 88:37-45 (1990); Charbit et al., Gene 70:181-189 (1988); Francisco et al., Proc. Natl. Acad. Sci. USA 89:2713-2717 (1992)), and yeast, such as Saccharomyces cerevisiae (Boder and Wittrup, Nat. Biotechnol. 15:553-557 (1997); Kieke et al., Protein Eng. 10:1303-1310 (1997)). Other known display techniques include ribosome or mRNA display (Mattheakis et al., Proc. Natl. Acad. Sci. USA 91:9022-9026 (1994); Hanes and Pluckthun, Proc. Natl. Acad. Sci. USA 94:4937-4942 (1997)), DNA display (Yonezawa et al., Nucl. Acid Res. 31(19):e118 (2003)); microbial cell display, such as bacterial display (Georgiou et al., Nature Biotech. 15:29-34 (1997)), display on mammalian cells, spore display (Isticato et al., J. Bacteria 183:6294-6301 (2001); Cheng et al., Appl. Environ. Microbiol. 71:3337-3341 (2005) and co-pending provisional application Ser. No. 60/865,574, filed Nov. 13, 2006), viral display, such as retroviral display (Urban et al., Nucleic Acids Res. 33:e35 (2005), display based on protein-DNA linkage (Odegrip et al., Proc. Acad. Natl. Sci. USA 101:2806-2810 (2004); Reiersen et al., Nucleic Acids Res. 33:e10 (2005)), and microbead display (Sepp et al., FEBS Lett. 532:455-458 (2002)).

At present, display of antibodies or antibody fragments on the surface of two types of bacteriophage, fd and M13, is the most widespread method for display, selection and engineering of antibodies. To express an antibody fragment on the surface of a phage particle, its coding sequence is fused in-frame to one of the phage coat proteins and cloned in a vector that can be packaged as a phage particle. Depending on the type of anchor protein and display vector, the display can be monovalent or multivalent. In phage display, selection of a library, such as a single-chain Fv (scFv) or Fab library, involves exposure of the library to antigen to allow antigen-specific phage antibodies to bind their target antigen during biopanning. This step is followed by recovery of antigen-bound phage and subsequent infection in bacteria. Typically, antibodies with the desired antigen-binding specificity are enriched by multiple rounds of selection. For further details of selecting and screening recombinant antibody libraries see, e.g., Hoogenboom, Nature Biotechnology 23(9): 1105-1116 (2005), and the references cited therein.

The DNA also may be modified, for example, by substituting the coding sequence for human heavy- and light-chain constant domains in place of the homologous murine sequences (U.S. Pat. No. 4,816,567; Morrison, et al., Proc. Natl. Acad. Sci. USA, 81:6851 (1984)), or by covalently joining to the immunoglobulin coding sequence all or part of the coding sequence for a non-immunoglobulin polypeptide.

(d) Humanized and Human Antibodies

A humanized antibody has one or more amino acid residues introduced into it from a source which is non-human. These non-human amino acid residues are often referred to as “import” residues, which are typically taken from an “import” variable domain. Humanization can be essentially performed following the method of Winter and co-workers (Jones et al., Nature, 321:522-525 (1986); Riechmann et al., Nature, 332:323-327 (1988); Verhoeyen et al., Science, 239:1534-1536 (1988)), by substituting rodent CDRs or CDR sequences for the corresponding sequences of a human antibody, although the technique of humanization has been significantly improved since these publications.

Thus, for example, SDR grafting, based on grafting, onto the human frameworks, only the specificity determining residues (SDRs), the CDR residues that are most crucial in the antibody-ligand interaction, is described by Kashmiri et al., Methods 36(1):25-34 (2005). The SDRs are identified through the help of a database of the three-dimensional structures of the antigen-antibody complexes of known structures or by mutational analysis of the antibody-combining site.

Antibody humanization by framework shuffling is described, for example, by Dall'Acqua et al., Methods 36(1):43-60 (2005).

It is also possible to produce transgenic animals (e.g., mice, rabbits, birds and cows) that are capable, upon immunization, of producing a full repertoire of human antibodies in the absence of endogenous immunoglobulin production. For example, the homozygous deletion of the antibody heavy-chain joining region (J_{H}) gene in chimeric and germ-line mutant mice results in complete inhibition of endogenous antibody production. Transfer of the human germ-line immunoglobulin gene array in such germ-line mutant mice will result in the production of human antibodies upon antigen challenge. See, e.g., Jakobovits et al, Proc. Natl. Acad. Sci. USA, 90:2551 (1993); Jakobovits et al., Nature, 362:255-258 (1993); Bruggermann et al., Year in Immunol., 7:33 (1993); and Duchosal et al. Nature 355:258 (1992). Human antibodies can also be derived from phage-display libraries (Hoogenboom et al, J. Mol. Biol., 227:381 (1991); Marks et al, J. Mol. Biol., 222:581-597 (1991); Vaughan et al. Nature Biotech 14:309 (1996)).

Surrogate Light Chain Constructs And SURROBODIES™

Constructs and libraries comprising surrogate light chain sequences and resulting binding polypeptides, referred to as “SURROBODIES™” are described in co-pending application Ser. No. 60/930,568 filed on Mar. 27, 2007, the entire disclosure of which is hereby expressly incorporated by reference.

Antibody (Ig) molecules produced by B-lymphocytes are built of heavy (H) and light (L) chains. The amino acid sequences of the amino terminal domains of the H and L chains are variable (V_{H} and V_{L}), especially at the three hypervariable regions (CDR1, CDR2, CDR3) that form the antigen combining site. The assembly of the H and L chains is stabilized by a disulfide bond between the constant region of the L chain (C_{L}) and the first constant region of the heavy chain (C_{HI}) and by non-covalent interactions between the V_{H} and V_{L} domains.

In humans and many animals, such as mice, the genes encoding the antibody H and L chains are assembled by stepwise somatic rearrangements of gene fragments encoding parts of the V regions. Various stages of B lymphocyte development are characterized by the rearrangement status of the Ig gene loci (see, e.g. Melchers, F. & Rolink, A., B-Lymphocyte Development and Biology, Paul, W. E., ed., 1999, Lippincott, Philadelphia).

Precursors of B cells (pre-B cells) have been identified in the bone marrow as lymphocytes that produce μ heavy chains but instead of the fully developed light chains express a set of B lineage-specific genes called VpreB(1-3) and λ5, respectively.

The main isoform of human VpreB1 (CAG30495) is a 145 aa-long polypeptide (SEQ ID NO: 1). It has an Ig V domain-like structure, but lacks the last β-strand (β7) of a typical V domain, and has a carboxyl terminal end that shows no sequence homologies to any other proteins. VpreB2 has several isoforms, including a 142-amino acid mouse VpreB2 polypeptide (P13373; SEQ ID NO: 2), and a 171 amino acids long splice variant of the mouse VpreB2 sequence (CAA019641 SEQ ID NO: 3). VpreB1 and VpreB2 sequences have been disclosed in EP 0 269 127 and U.S. Pat. No. 5,182,205; Collins et al., Genome Biol. 5(10):R84 (2004); and Hollins et al., Proc. Natl. Acad. Sci. USA 86(14):5552-5556 (1989). The main isoform of human VpreB3 (SEQ ID NO: 4) is a 123 aa-long protein (CAG30496), disclosed in Collins et al., Genome Biol. 5(10):R84 (2004).

VpreB(1-3) are non-covalently associated with another protein, λ5. The human λ5 is a 209-amino acid polypeptide (CAA01962; SEQ ID NO: 5), that carries an Ig C domain-like structure with strong homologies to antibody light chains and, towards its amino terminal end, two functionally distinct regions, one of which shows strong homology to the β7 strand of the Vλ domains. A human λ5-like protein has 213 amino acids (NP_{—}064455; SEQ ID NO: 6) and shows about 84% sequence identity to the antibody λ light chain constant region.

For further details, see the following review papers: Karasuyama et al., Adv. Immunol. 63:1-41 (1996); Melchers et al., Immunology Today 14:60-68 (1993); and Melchers, Proc. Natl. Acad. Sci. USA 96:2571-2573 (1999).

The VpreB and λ5 polypeptides together form a non-covalently associated, Ig light chain-like structure, which is called the surrogate light chain or pseudo light chain. On the surface of early preB cells, the surrogate light chain is disulfide-linked to membrane-bound Ig μ heavy chain in association with a signal transducer CD79a/CD79b heterodimer to form a B cell receptor-like structure, the so-called preB cell receptor (preBCR).

The polypeptides, including bispecific polypeptides, of the present invention may comprise VpreB sequences comprising a first binding region having the ability to bind a target. The target can be any peptide or polypeptide that is a binding partner for the VpreB sequence-containing polypeptides of the present invention. Targets specifically include all types of targets generally referred to as “antigens” in the context of antibody binding. In the VpreB-containing bispecific polypeptides of the present invention, the second binding region may be provided by a second VpreB sequence or by a heterogeneous amino acid sequence associated with the sequence providing the first VpreB binding region. Association may be either covalent or non-covalent, and may occur directly, or through a linker, including peptide linkers.

If desired, the surrogate light chain-containing constructs, or Surrobodies, of the present invention can be engineered, for example, by incorporating or appending known sequences or sequence motifs from the CDR1, CDR2 and/or CDR3 regions of antibodies, including known therapeutic antibodies into the CDR1, CDR2 and/or CDR3 analogous regions of the surrogate light chain sequences. This allows the creation of molecules that are not antibodies, but will exhibit binding specificities and affinities very similar to those of a known therapeutic antibody.

All surrogate light chain constructs may be associated with antibody sequences, which, in certain embodiments, provide the second specificity. For example, a VpreB-λ5 fusion can be linked to an antibody heavy chain variable region sequence by a peptide linker. In another embodiment, a VpreB-λ5 fusion is non-covalently associated with an antibody heavy chain, or a fragment thereof including a variable region sequence to form a dimeric complex. In yet another embodiment, the VpreB and λ5 sequences are non-covalently associated with each other and an antibody heavy chain, or a fragment thereof including a variable region sequence, thereby forming a trimeric complex. In all of these constructs, the second binding region may, for example, bind to the first binding region or to a framework region in another molecule of the surrogate light chain-containing bispecific polypeptide. In a further embodiment, the second binding region will only bind to a complex formed between the surrogate light chain-containing bispecific polypeptide and a target.

Bispecific Polypeptides

The present invention includes bispecific polypeptides, such as bispecific antibodies and their uses. As discussed earlier, the term “bispecific” is used in the broadest sense and includes molecules, such as polypeptides, e.g. antibodies with more than one specificity, i.e. tri- and multi-specific polypeptides, e.g. antibodies.

In one aspect, the bispecific polypeptides are bispecific antibodies or bispecific antibody fragments. Bispecific polypeptides and bispecific antibodies and antibody fragments, in general, are known in the art. Bispecific antibody fragments include, for example, diabodies in which V_{H} and V_{L} domains are expressed on a single polypeptide chain, but using a linker that is too short to allow for pairing between the two domains on the same chain, thereby forcing the domains to pair with complementary domains of another chain and creating two antigen binding sites (see e.g., Holliger, P., et al., Proc. Natl. Acad. Sci. USA 90:6444 6448 (1993), and Poljak, R. J., et al., Structure 2:1121 1123 (1994)). Bispecificity can also be achieved by fusing two single-chain Fv (scFv) or Fab via flexible linkers (Mallender and Voss, J. Biol. Chem., 269, 199-206 (1994); Mack et al., Proc. Natl. Acad. Sci. USA, 92, 7021-7025 (1995); Zapata et al., Protein Engng, 8, 1057-1062 (1995)), leucine zipper (De Kruif and Logtenberg, Biol. Chem., 271, 7630-7634 (1996), C_{H}C_{L}-heterodimerization domain (Müller et al., FEBS Lett. 422(2):259-64 (1998); and diabody format (Holliger et al., Proc. Natl. Acad. Sci. USA 90(14):6444-8 (1993). Bispecific antibodies and other bispecific polypeptides, such as bispecific immunoadhesins, can also be made by the “knobs-into-holes” method, described, for example, by Ridgeway and Presta, Protein Engineering 9(7):617-21 (1996).

As discussed earlier, the bispecific polypeptides, including bispecific antibodies and antibody fragments herein, are characterized by having one binding region binding to a target, e.g. a target antigen, and a second binding region binding to another molecule of the bispecific polypeptide, such as a bispecific antibody or antibody fragment.

In one embodiment, the second binding region shows binding specificity for the first binding region. Thus, for example, in the case of an antibody or antibody fragment, the first binding region recognizes the target antigen and the second binding region recognizes the idiotype of the first binding region (anti-idiotypic antibody). Thus, the second binding region of the antibody or antibody fragment competes with the antigen for binding to the first binding region. As a result, a multi-linked antibody complex is formed, which is attached to the target antigen solely through a single first binding region (variable region sequence) of the bispecific antibody. However, this single attachment brings along multiple Fc regions as part of the multi-linked antibody complex, which results in improved effector functions, such as additional antibody-dependent cellular cytotoxicity (ADCC) and complement-dependent cytotoxicity (CDC) capacities. CDC results from C1q binding, and ADCC is mediated by one or more of the Fcγ receptors on the surface of granulocytes, macrophages and NK cells. For further details of antibody effector functions, and in particular ADCC and CDC see, for example, the following review articles: Daeron, Annu. Rev. Immunol. 15:203 234 (1997); Ward and Ghetie, Therapeutic Immunol. 2:77 94 (1995); and Ravetch and Kinet, Annu. Rev. Immunol. 9:457 492 (1991).

ADCC and/or CDC have been shown to play a major role in the mechanism of action of several therapeutic antibodies, such as HERCEPTIN® (trastuzumab, Genentech, Inc.). Thus, enhancement of antibody effector functions, such as ADCC and/or CDC, is expected to result in improved efficacy in the treatment of viral infections, cancer therapy, and other indication areas. As a result, the multi-linked antibody complex formed by the binding of this type of the bispecific antibodies of the present invention will be more effective in activating the immune system and clearing the target antigen, such as a viral or tumor antigen.

In another embodiment, just as before, the first binding region of a bispecific antibody or antibody fragment binds to the target antigen, while the second binding region binds, in another molecule of the same bispecific antibody or antibody fragment, to the framework of the sequence (arm) carrying the first binding region. Since the second binding region does not bind to the first binding region and thus does not compete for binding to the target antigen, in this case, the multi-linked antibody complex formed will attach to the target antigen through multiple linkages. This is expected to create a highly avid and potentially functionally non-dissociative interaction with the target antigen, thereby increasing clearance of the target antigen.

In yet another embodiment, the first binding region of a bispecific polypeptide recognizes the target and the second binding region recognizes only the complex formed between the bispecific polypeptide and the target, as a result of binding through the firm binding region. In this instance the reaction proceeds only in the presence of the target, such as a target antigen in the case of a bispecific antibody or antibody fragment.

The bispecific (including multi-specific) polypeptides herein specifically include bispecific (including multi-specific) surrogate light chain constructs. Specific examples of the surrogate light chain-containing bispecific polypeptides herein include polypeptides in which a VpreB sequence, such as a VpreB1, VpreB2, or VpreB3 sequence, including fragments and variants of the native sequences, is conjugated to a λ5 sequence, including fragments and variants of the native sequence, to provide the first binding region. In a direct fusion, typically the C-terminus of a VpreB sequence (e.g. a VpreB1, VprB2 or VpreB3 sequence) is fused to the N-terminus of a λ5 sequence. While it is possible to fuse the entire length of a native VpreB sequence to a full-length λ5 sequence, typically the fusion takes place at or around a CDR3 analogous site in each of the two polypeptides. In a preferred embodiment, the fusion takes place between about amino acid residues 116-126 of the native human VpreB1 sequence (SEQ ID NO: 1) and between about amino acid residues 82 and 93 of the native human λ5 sequence (SEQ ID NO: 5).

It is also possible to fuse the VpreB sequence to the CDR3 region of an antibody λ light chain in a construct providing the first binding region. Further constructs, in which either or both VpreB and λ5 is truncated, and similar constructs using antibody κ light chain sequences are also possible and may be included in the surrogate-light chain containing bispecific polypeptides herein.

Further details of the invention are provided in the following non-limiting Examples.

### Example 1

### Selection of Anti-Framework Antibodies

For selecting antibodies binding to the framework of the antibody binding to the target antigen (briefly referred to as “anti-framework antibodies”) two targets are used: an antigen specific antibody Fab fragment (Fab 1) and the corresponding germline antibody Fab fragment (Fab1germ). Panning is performed in 8 wells of a 96-well ELISA plate that have been coated with 100 ng/well anti-Hemagglutinin Fab (Fab1) and blocked with 3% nonfat milk in PBS-Tween (0.05%). After blocking, the plate is washed three times with PBS-Tween (0.05%) after which Fab1 is ready for panning. Thereafter, 0.1 ml of a previously blocked antibody repertoire are added to each well, followed by incubation for 2 hours at 4 degrees. The wells are then washed 10-12 times and then eluted at low pH, neutralized and then amplified for another round of panning. In the second round, a similar procedure is followed, but the panning is performed with the germline Fab (Fab1germ). The secondary selection reduces the occurrence of CDR- or Fv-specific Fabs. Subsequent rounds alternate between these two selections. Alternation of targets is an important step for removing anti-CDR and anti-Fv antibodies and enriching highly specific anti-framework antibodies.

Alternatively, the first step is panning Fab1germ, followed by subsequent rounds utilizing individual combinatorial targets of family-related germline frameworks. This variant more broadly identifies anti-framework antibodies for familial groups.

In either instance, either type of antibody can be used to crosslink antigen specific antibodies prior to, or after antigen binding. Furthermore, either approach can be used to produce bispecific antibodies that generate synthetic “opsonizing” nets to capture a desired target.

### Example 2

### Selection of Anti-Fv Antibodies

In this method, it is first necessary to remove anti-framework antibodies. To do so 1 ml of the antibody repertoire is incubated with 0.05 ml dynal streptavidin magnetic beads previously coated with a biotinylated germline antibody Fab fragment (Fab1germ), previously blocked in 3% nonfat milk in PBS-Tween (0.05%). The incubation proceeds for 1 hour at 4 degrees and the beads are separated and the phage supernatant is recovered. The anti-framework phage are then placed into 8 wells of a 96-well ELISA plate that have been coated with 100 ng/well anti-Hemagglutinin Fab (Fab1) and blocked with 3% nonfat milk in PBS-Tween (0.05%). After blocking, the plate is washed three times with PBS-Tween (0.05%) and the Fab is now ready for panning. 0.1 ml of the previously blocked antibody repertoire are added to each well, followed by incubation 2 hours at 4 degrees. The wells are then washed 10-12 times and eluted at low pH, neutralized and amplified for another round of panning. In the second round, a similar procedure is followed, first subtracting the amplified phage with Fab1germ and then selecting on the anti-hemagglutinin Fab1. This selection should reduce the occurrence of framework specific antibodies and produce anti-Fv antibodies.

Alternatively framework subtracting can be performed with Fabs of all the related germlines prior to Fv panning. This approach more specifically identifies anti-Fv antibodies for familial groups.

Such antibodies can be used to crosslink antigen specific antibodies that process away from the physical target and provide somewhat of a protruding and repetitive Fc presenting structure. In this instance single or few points of surface contact are made, as the second arm or antibody continues to displace the first, until it is emerged from the surface. This emerging structure becomes an “opsonizing” beacon for immune clearance.

### Example 3

### Selection of Anti-Complex Antibodies

To create a bispecific chain reactive antibody one first needs an antigen (e.g., Hemagglutinin or “HA”) specific antibody (FAb1) and secondly an antibody that recognizes the first antibody as it is complexed to its cognate antigen (FAb2). To isolate and enrich such antibodies that recognize a complex of proteins the antibodies recognizing the individual components need to be reduced or eliminated. According to one approach, from an antibody repertoire the antibodies that bind the unliganded antigen specific antibody (mAb1) are subtracted. To do so 1 ml of the antibody repertoire is incubated with 0.05 ml dynal streptavidin magnetic beads previously coated with biotinylated FAb1 and blocked in 3% nonfat milk in PBS-Tween (0.05%). The incubation proceeds for 1 hour at 4 degrees and the beads are separated and the phage supernatant is recovered. Next the phage antibodies are incubated with 0.05 ml dynal streptavidin magnetic beads previously coated with biotinylated HA protein and blocked in 3% nonfat milk in PBS-Tween (0.05%). The incubation proceeds similarly for 1 hour at 4 degrees and the beads are separated and the phage supernatant is recovered. The next step is positive selection or “panning.”

Panning is performed in 8 wells of an 96-well ELISA plate that have been coated with 100 ng/well Hemagglutinin protein and blocked with 3% nonfat milk in PBS-Tween (0.05%). Next 100 ng of Fab1 in 0.1 ml blocking buffer is incubated for 1 hour at 4 degrees. After blocking, the plate is washed three times with PBS-Tween (0.05%) and the complex is now ready for panning. 0.1 ml of the subtracted antibody repertoire is added to each well and the wells are incubated for 2 hours at 4 degrees. The wells are then washed 10-12 times, eluted at low pH, neutralized and then amplified for another round of palming. In the second round, a similar procedure is followed, but the panning orientation is reversed. Thus, Fab is immobilized first, then blocked, and then HA protein is incubated and bound, prior to phage panning. Subsequent rounds alternate between these two orientations. Alternation of orientation is an important step in removing non-complex recognizing antibodies and enriching for complex specific antibodies. In total this selection process typically requires about 6-10 rounds of selection.

The resultant antibodies can be used to crosslink antigen specific antibodies until the antigen becomes exhausted, providing a target-dependent and self-assembling “opsonizing” net for immune clearance. This process can be further used to select for any complex-specific antibodies by simply substituting Fab1 and HA with two subunits of a heteromeric protein or any other two-piece complexes, proteinaceous or otherwise.

### Example 4

### Multi-Specific Anti-Complex Antibodies

To create a multispecific chain reactive antibody one first needs an antigen (ErbB2) specific antibody (mAb1—“Trastuzumab”) and secondly an antibody that recognizes the first antibody as it is complexed to its' cognate antigen (FAb2). Furthermore it is necessary to have a second antigen specific antibody (FAb3) that recognizes CD16. To isolate and enrich such antibodies that recognize a complex of proteins one needs to reduce or eliminate those antibodies recognizing the individual components. In the present case, antibodies that bind the unliganded antigen specific antibody (mAb1) need to be subtracted from an antibody repertoire. To do so 1 ml of the antibody repertoire is incubated with 0.05 ml dynal streptavidin magnetic beads previously coated with biotinylated FAb1 and blocked in 3% nonfat milk in PBS-Tween (0.05%). The incubation proceeds for 1 hour at 4 degrees and the beads are separated and the phage supernatant is recovered. Next the phage antibodies are incubated with 0.05 ml dynal Protein A magnetic beads previously coated with ErbB2-Fc protein fusion and blocked in 3% nonfat milk in PBS-Tween (0.05%). The incubation proceeds similarly for 1 hour at 4 degrees and the beads are separated and the phage supernatant is recovered. At this point, the subtracted antibody repertoire is ready for positive selection or “panning.”

Panning is performed in 8 wells of an 96-well ELISA plate that have been coated with 100 ng/well erbB2-Fc protein and blocked with 3% nonfat milk in PBS-Tween (0.05%).

Next 100 ng of Fab1 in 0.1 ml blocking buffer is incubated for 1 hour at 4 degrees. After blocking, the plate is washed three times with PBS-Tween (0.05%) and the complex is now ready for panning. 0.1 ml of the subtracted antibody repertoire is added to each well and incubated for 2 hours at 4 degrees. The wells are then washed 10-12 times and eluted at low pH, neutralized and then amplified for another round of panning. In the second round proceeds with a similar procedure, but the panning orientation is reversed, meaning the Fab is immobilized first, then blocked, and then ErbB2-Fc protein is incubated and bound, prior to phage panning. Subsequent rounds alternate between these two orientations. Alternation of orientation is an important step to removing non-complex recognizing antibodies and enriching for complex specific antibodies. Usually 6-10 rounds of selection are performed.

The obtained anti-complex antibody is then utilized to make a bispecific antibody composed of an anti-CD 16 antibody arm and an anti-erbB2/Transtuzumab complex antibody arm. This multi-antigen complex can more effectively recruit immune effector function thereby driving more cancer cell killing.

Furthermore this process can be used to select for any complex specific antibodies by simply substituting Fab1 and HA or erbB2 with two subunits of a heteromeric protein or any other two piece complexes, proteinaceous or otherwise.

Although in the foregoing description the invention is illustrated with reference to certain embodiments, it is not so limited. Indeed, various modifications of the invention in addition to those shown and described herein will become apparent to those skilled in the art from the foregoing description and fall within the scope of the appended claims.

All references cited throughout the specification, and the references cited therein, are hereby expressly incorporated by reference in their entirety.

1. A method for preparing an oligomer comprising repeats of a first and a second binding polypeptide bound to a target, comprising contacting molecules of
(a) said first binding polypeptide having binding specificity for said target and said second binding polypeptide having binding specificity for said first binding polypeptide, or
(b) said first binding polypeptide having binding specificity for a target and said second binding polypeptide having binding specificity for a complex formed between said first binding polypeptide and said target,
under conditions that restrict intramolecular or bimolecular binding and allow intermolecular or greater than bimolecular binding, such that a processive intermolecular chain reaction between molecules of the first and second binding polypeptides occurs,
wherein at least one molecule of said first binding polypeptide binds to said target before or after said chain reaction,
whereby an oligomer comprising repeats of said first and second binding polypeptides attached to said target at least one point is formed.

2. The method of claim 1 wherein said first and said second binding polypeptides are selected from the group consisting of antibodies, antibody fragments, surrogate light chain constructs, immunoadhesins, receptors, ligands, and enzymes.

3. The method of claim 2 wherein said first and said second binding polypeptides are antibodies or antibody fragments and said target is an antigen.

4. The method of claim 3 wherein said antibody fragment is selected from the group consisting of Fab, Fab′, F(ab′)₂, scFv, (scFv)₂, dAb, and complementarity determining region (CDR) fragments, linear antibodies, single-chain antibody molecules, minibodies, diabodies, and multispecific antibodies formed from antibody fragments.

5. The method of claim 3 wherein the second antibody or antibody fragment binds to the framework region of the first antibody or antibody fragment.

6. The method of claim 5 wherein the oligomer formed is attached to the antigen at more than one point.

7. The method of claim 3 wherein the second antibody or antibody fragment binds to the antigen-binding region of the first antibody or antibody fragment.

8. The method of claim 7 wherein the oligomer formed is attached to the antigen at one point.

9. The method of claim 3 wherein the second antibody or antibody fragment binds to the complex formed between the first antibody or antibody fragment and the antigen.

10. The method of claim 9 wherein the oligomer formed is attached to the antigen at more than one point.

11. A method for preparing an oligomer comprising repeats of a binding polypeptide having at least a first and second binding specificity, bound to a target, comprising reacting molecules of said binding polypeptide under conditions that restrict intramolecular or bimolecular binding and allow intermolecular, or greater than bimolecular binding such that a processive intermolecular chain reaction between molecules of said binding polypeptide occurs, wherein
(a) said first binding specificity is for a target and said second binding specificity is for another molecule of said binding polypeptide, or
(b) said first binding specificity is for a target and said second binding specificity is for a complex formed between said binding polypeptide and said target,
wherein at least one molecule of said binding polypeptide binds to said target before or after said chain reaction, and
whereby an oligomer comprising repeats of said binding polypeptide attached to said target is formed.

12. The method of claim 11 wherein said binding polypeptide is selected from the group consisting of antibodies, antibody fragments, surrogate light chain constructs, immunoadhesins, receptors, ligands, and enzymes.

13. The method of claim 12 wherein said binding polypeptide is an antibody or an antibody fragment, and said target is an antigen.

14. The method of claim 13 wherein said antibody fragment is selected from the group consisting of Fab, Fab′, F(ab′)₂, scFv, (scFv)₂, dAb, and complementarity determining region (CDR) fragments, linear antibodies, single-chain antibody molecules, minibodies, diabodies, and multispecific antibodies formed from antibody fragments.

15. The method of claim 13 wherein the second binding specificity of said antibody or antibody fragment is for the framework region of another molecule of said antibody or antibody fragment.

16. The method of claim 15 wherein the oligomer formed is attached to the antigen at more than one point.

17. The method of claim 13 wherein the second binding specificity of said antibody or antibody fragment is for the antigen-binding region of another molecule of said antibody or antibody fragment.

18. The method of claim 17 wherein the oligomer formed is attached to the antigen at one point.

19. The method of claim 13 wherein the second binding specificity of said antibody or antibody fragment is for the complex formed between another molecule of said antibody or antibody fragment and the antigen.

20. The method of claim 19 wherein the oligomer formed is attached to the antigen at more than one point.

21. A bispecific polypeptide comprising a first binding region binding to a target and a second binding region recognizing and binding to a sequence within another molecule of the same bispecific polypeptide.

22. The bispecific polypeptide of claim 21 which is a bispecific antibody or a bispecific antibody fragment.

23. The bispecific polypeptide of claim 22 wherein the first binding region of said bispecific antibody or antibody fragment binds to a target antigen and the second binding region recognizes and binds to the first binding region of another molecule of the same bispecific antibody or antibody fragment.

24. The bispecific polypeptide of claim 22 wherein the first binding region of said bispecific antibody or antibody fragment binds to a target antigen and the second binding region binds to a framework sequence of another molecule of the same bispecific antibody or antibody fragment.

25. The bispecific polypeptide of claim 22 wherein the first binding region of said bispecific antibody or antibody fragment binds to a target antigen and the second binding region binds to another molecule of the same bispecific antibody or antibody fragment only when said molecule is in the form of a complex with said target antigen.

26. The bispecific polypeptide of claim 21 comprising at least one surrogate light chain sequence.

27. The bispecific polypeptide of claim 26 additionally comprising an antibody sequence.

28. The bispecific polypeptide of claim 21 comprising a ligand binding sequence of a receptor.

29. The bispecific polypeptide of claim 21 comprising a receptor binding sequence of a ligand.

30. The bispecific polypeptide of claim 21 which is a bispecific immunoadhesin or a fragment thereof.

31. An oligomer comprising repeats of a first and a second binding polypeptide bound to a target at least one site.

32. The oligomer of claim 31 wherein said first and said second binding polypeptides are identical.

33. The oligomer of claim 31 wherein said first and said second binding polypeptides are different.

34. The oligomer of claim 31 which is bound to said target at more than one site.

35. The oligomer of claim 31 comprising at least two repeats of said first and said second binding polypeptides.

36. The oligomer of claim 35 comprising two to 4 repeats of said first and said second binding polypeptides.

37. The oligomer of claim 31 wherein said first and said second binding polypeptides are selected from the group consisting of antibodies, antibody fragments, surrogate light chain constructs, immunoadhesins, receptors, ligands, and enzymes.

38. The oligomer of claim 36 wherein said first and said second binding polypeptides are antibodies or antibody fragments and said target is an antigen.

39. The oligomer of claim 38 wherein said antibody fragment is selected from the group consisting of Fab, Fab′, F(ab′)₂, scFv, (scFv)₂, dAb, and complementarity determining region (CDR) fragments, linear antibodies, single-chain antibody molecules, minibodies, diabodies, and multispecific antibodies formed from antibody fragments.

40. A composition comprising a bispecific polypeptide according to claim 21 or an oligomer according to claim 31 in admixture with a carrier.

41. The composition of claim 40 which is a pharmaceutical composition.

42. A method for the prevention or treatment of a disease or condition benefiting from the enhancement of immune response comprising administering to a mammalian patient in danger of developing or having said disease or condition an effective amount of an oligomer of claim 31.

43. The method of claim 42 wherein said disease or condition is a B cell neoplasm.

44. The method of claim 43 wherein said B cell neoplasm is a B cell lymphoma.

45. The method of claim 44 wherein said B cell lymphoma is selected from the group consisting of a non-Hodgkin's lymphoma (NHL); follicular center cell (FCC) lymphoma, acute lymphocytic leukemia (ALL), chronic lymphocytic leukemia (CLL), and Hairy cell leukemia.

46. The method of claim 45 wherein said non-Hodgkins lymphoma is selected from the group consisting of low grade/follicular non-Hodgkin's lymphoma (NHL), small lymphocytic (SL) NHL, intermediate grade/follicular NHL, intermediate grade diffuse NHL, high grade immunoblastic NHL, high grade lymphoblastic NHL, high grade small non-cleaved cell NHL, bulky disease NHL, plasmacytoid lymphocytic lymphoma, mantle cell lymphoma, AIDS-related lymphoma and Waldenstrom's macroglobulinemia.

47. The method of claim 42 wherein said disease or condition is cancer.

48. The method of claim 47 wherein said cancer is breast cancer.

49. The method of claim 48 wherein said cancer is metastatic breast cancer.
